(19) United States
(12) Plant Patent Application Publication
(10) Pub. No.: US 2014/0352013 P1
(43) Pub. Date: Nov. 27, 2014

(54) Maple tree (Acer truncatum) variety named BABY DRAGON

(50) ;

(71) Applicant: KEITH G. JOHANSSON, Arlington, TX (US)

(72) Inventor: KEITH G. JOHANSSON, Arlington, TX (US)

(21) Appl. No.: 13/815,604

(22) Filed: May 22, 2013

(51) Int. Cl. A01H 5/00

(52) U.S. Cl. PLT/224

(57) ABSTRACT

A novel variety of the maple tree Acer truncatum was discovered in North Texas. It has a distinct dwarf size and prolific, very small leaves which weigh down its thin, willowy branches, giving it a weeping bushy habit. It exhibits distinctive, pink new growth which persists throughout spring and summer and into fall, whereupon it develops striking, deep yellow and red fall colors. The variety known as ‘Baby Dragon’ also is highly tolerant to direct summer sun and low-light conditions alike, as well as drought conditions, especially for a dwarf variety.

### LATIN NAME OF THE GENUS AND SPECIES AND THE VARIETY DENOMINATION OF THE PLANT CLAIMED

Acer truncatum ‘Baby Dragon’

### BACKGROUND OF THE INVENTION

1. Field of the Invention

This invention relates generally to decorative maple trees known by the scientific name Acer truncatum and the common name “Shantung” maple, and particularly to a new and distinct variety thereof identified as Acer truncatum ‘Baby Dragon’. I discovered it growing in a cultivated area in my nursery in Fort Worth, Tex., in 1994. I have given it the name ‘Baby Dragon’ because of its dwarf size and miniature leaves.

No federally sponsored research or development was involved in this invention.

2. Background

Maple trees provide desirable shade from spring to fall and typically shed their leaves for winter, thereby providing useful surrounding vegetation where summer sunlight may be excessive and winter sunshine is desirable. Displaying striking colors, maple trees are characteristically ornamental and provide pleasing and varying visual effects throughout their foliage period.

The Acer truncatum maple tree, commonly called “Shantung” maple, is a newly introduced tree in the United States which does exceptionally well in the Midwest in hot climates and heavily alkaline soils. Shantung maples generally are characterized by having truncated base lobes near the leaf petiole and by having a prominent central lobe, giving the leaf a distinctly ovate-truncate shape. Fall foliage appears yellow, with occasional areas of lesser reds or orange mixed with yellow, though the latter seldom is repeated in subsequent years.

The instant novel variety of maple tree now discovered is a dwarf variety with small leaves that is ideally suited for rock gardens and foundation planting. Baby Dragon maples have thin, willowy branches that give it a weeping, bushy appearance. It always seems to be growing vigorously, but never gets big. The original tree is seventeen years old and only 4′-10″ tall. The tree is a distinctive dwarf maple that should grow to only two (2) meters or so in height and have a dense, weeping, rounded crown with a greater spread than height.

The instant novel variety of maple tree also exhibits vibrant spring and summer colors and its summer colors include no green new growth as typical of many Shantung maples. A Baby Dragon maple's new growth is more pink and exhibits a rare, strong pink in summer, but never the purplish-red of most Shantung maples. It also exhibits deep yellow and red fall colors similarly to Shantung, but produces more consistent mixed fall colors than typical.

The instant novel variety of maple tree also is a very adaptable and hardy tree that withstands full sun in the Midwest and lower South, whereas other dwarf (typically Japanese) maples easily burn in direct summer sunlight, even in northern climates.

### SUMMARY OF THE INVENTION

The novel cultivar of the present invention, named Acer truncatum ‘Baby Dragon,’ is believed to be a variant of Acer truncatum distinguished by its dwarf size, willowy weeping bushy habit, good heat and sun tolerance, and a consistent pink new growth color throughout the spring and summer, developing striking deep yellow and red coloration variations in the fall. Specifically:

(a) its dwarf stature results in a small tree ideally suited for gardens and foundation growth;

(b) its very small but prolific leaves weigh down its thin, willowy branches, leading to a weeping appearance;

(c) its distinctive pink new growth persists throughout spring and summer and into fall, whereupon it develops deep yellow and red fall colors; and

(d) is highly tolerant to direct summer sun and low-light conditions alike, as well as drought conditions, especially for a dwarf variety.

### BRIEF DESCRIPTION OF THE DRAWINGS

FIGS. 1A-1B are photographic views of mature original trees, showing the willowy, weeping bushy habit during summer foliage.

FIG. 2A is a photographic close-up view of the trunk and low major branches of the mature original tree of FIG. 1.

FIG. 2B is a photographic close-up of the trunk of the mature original tree of FIG. 1A and showing bark characteristics.

FIG. 2C is a photographic view of the mature original tree of FIG. 1A and exhibiting its willowy, thin branch structure.

FIGS. 3A and 3B are photographic close-up views of lateral and terminal buds, respectively, of the mature original tree of FIG. 1A.

FIG. 4A is a photographic close-up of a branch of growth leaves on the mature original tree of FIG. 1A, illustrating the size (juxtaposed to a man's hand) and vigorous profusion of leaves on a typical branch of the mature original tree of FIG. 1A.

FIG. 4B is a photographic view of individual leaves of the mature original tree of FIG. 1A and showing actual size against a measuring tape.

FIGS. 5A-5C are photographic distance and close-up views of the early flush deep pink coloration of new growth on the mature original tree of FIG. 1A.

FIGS. 6A and 6B are photographic distance and close-up views of the particularly striking strong pink of summer growth on the mature original tree of FIG. 1A.

FIGS. 7A and 7B are photographic distance and close-up views of the deep yellow and red fall colors of the mature original tree of FIG. 1A.

### DETAILED DESCRIPTION OF THE INVENTION

The instant variety of maple tree was discovered as a seedling growing among a group of other maple tree seedlings of the Acer truncatum species at my nursery in Fort Worth, Tex., in 1994. It was successfully asexually reproduced at said nursery by grafting onto other Acer truncatum seedlings and by semi-hardwood cuttings. The novel characteristics are reliably transmitted to succeeding generations.

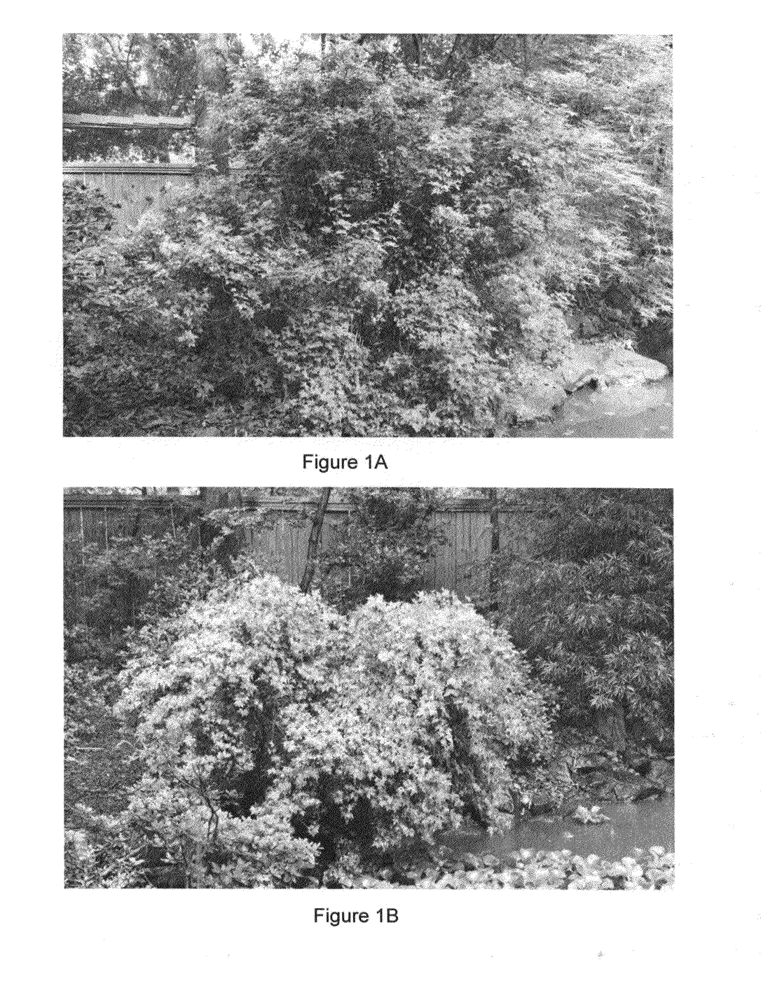

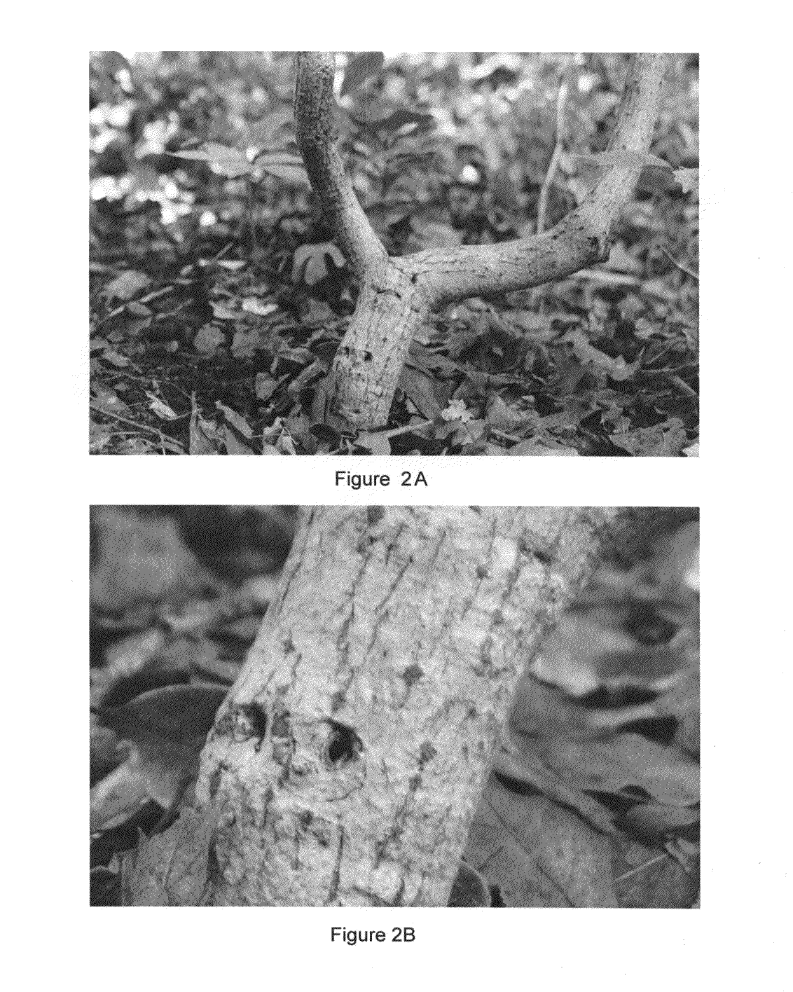

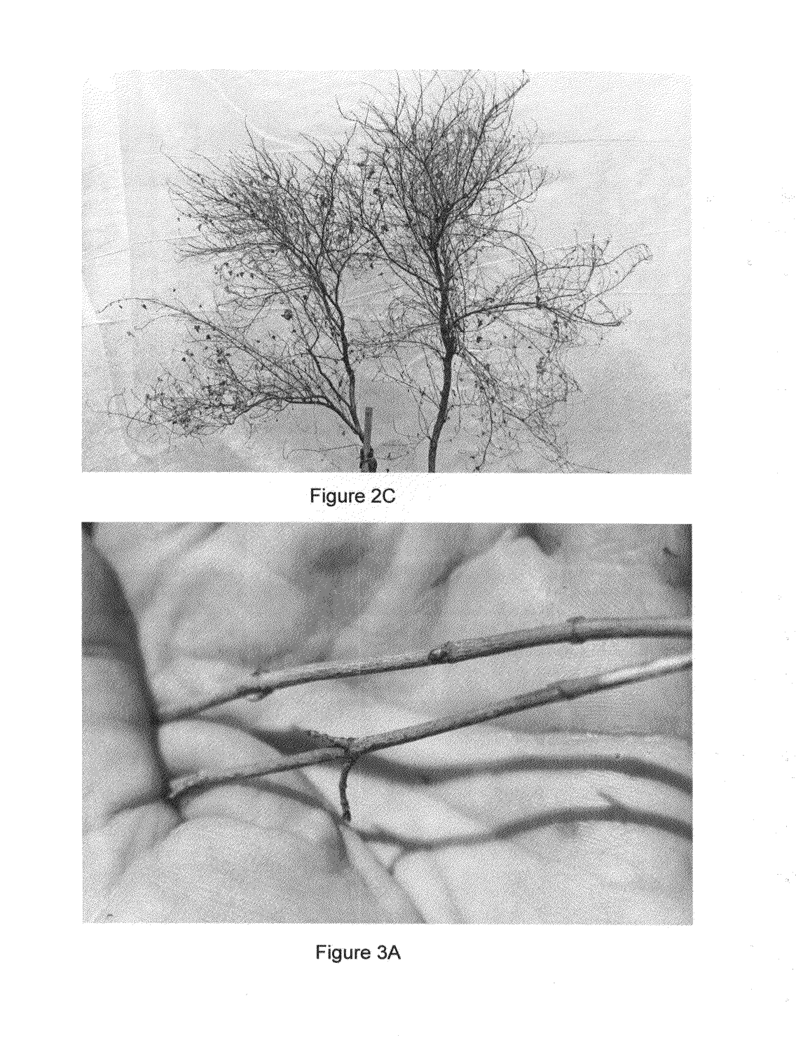

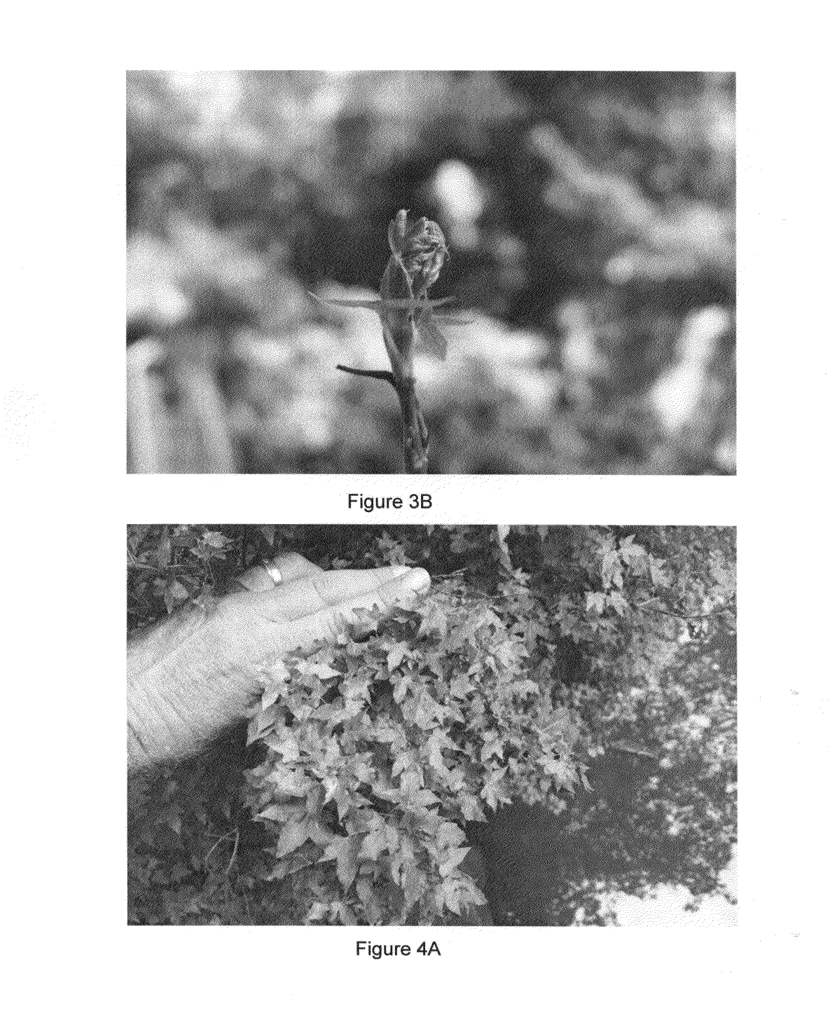

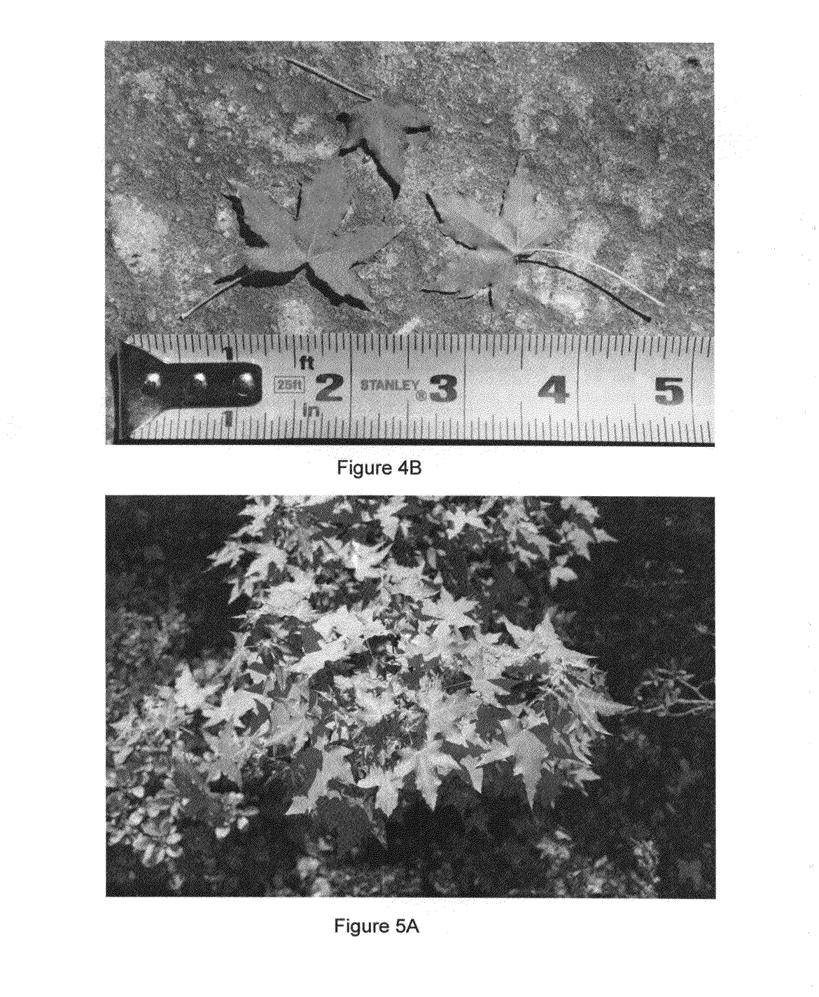

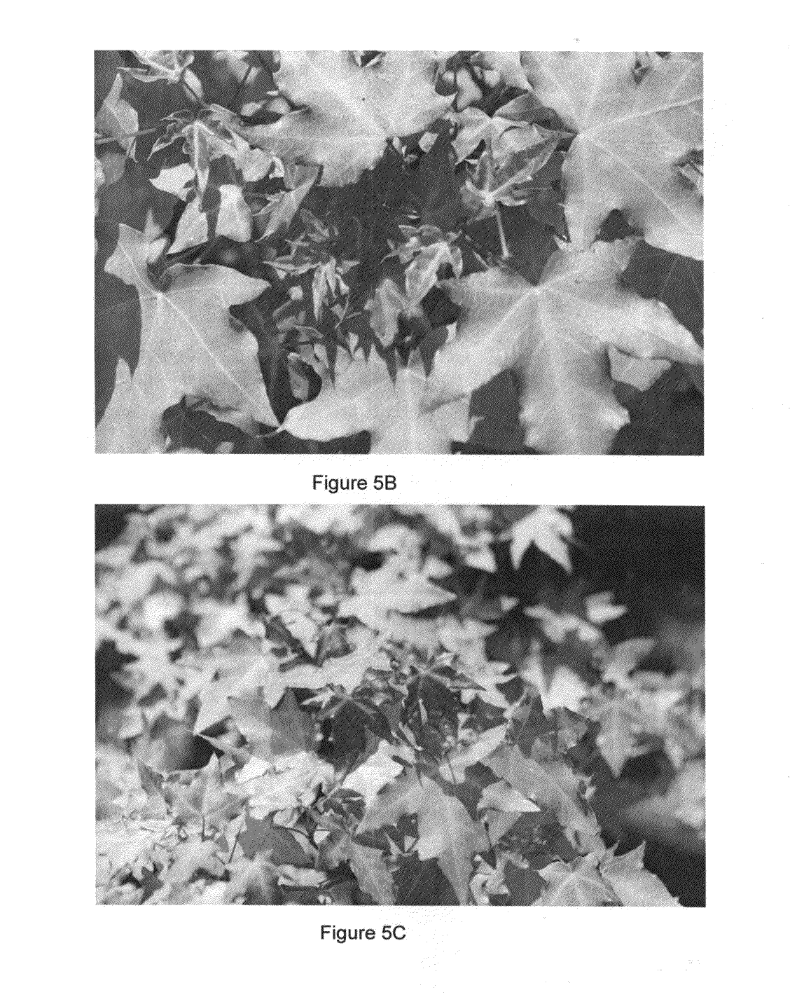

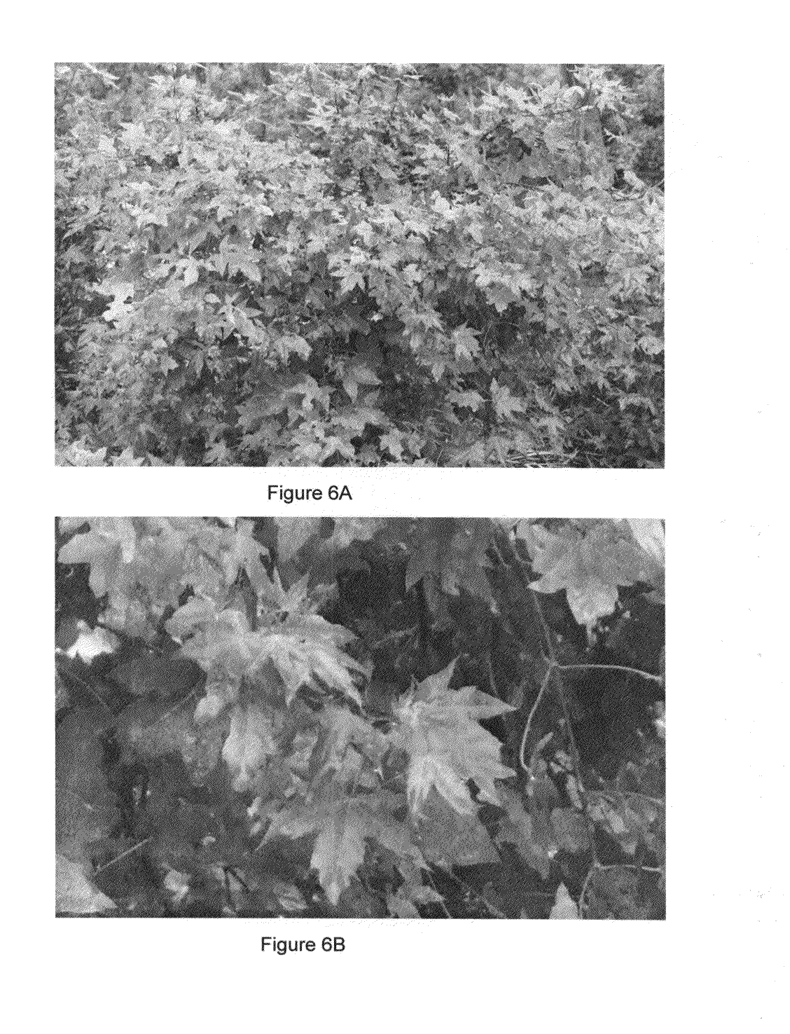

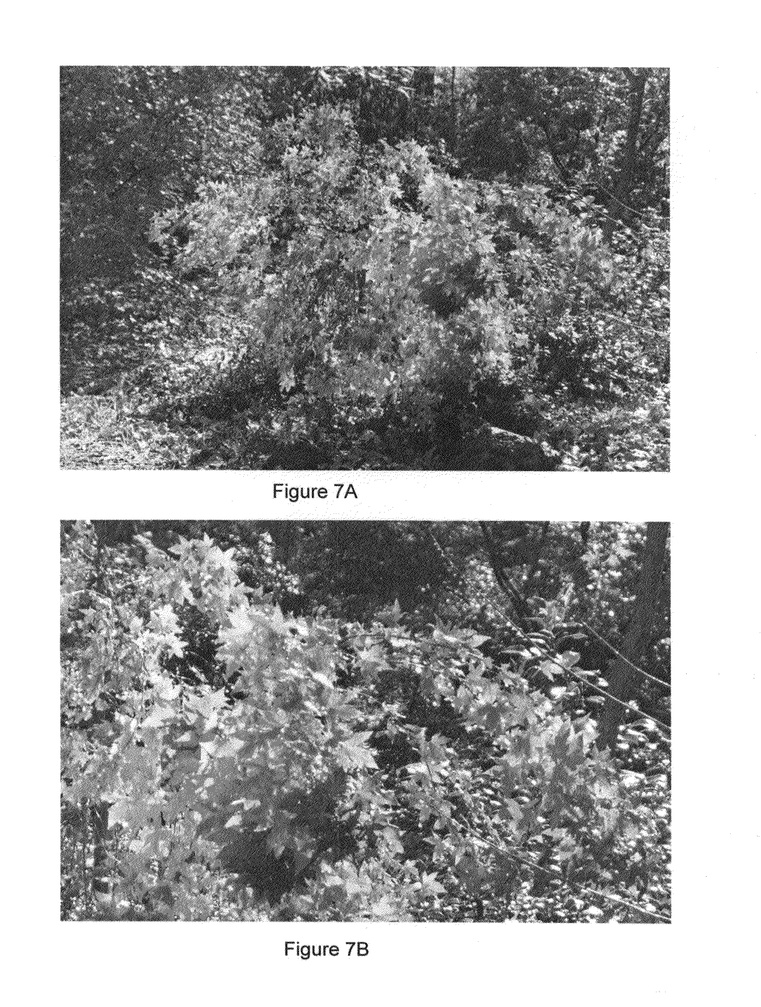

With reference to the seventeen (17 y.o.) year old trees depicted in the figures, the following specific description of the instant maple variety details its unique characteristics. Color terminology refers to The 2001 Royal Horticultural Society (R.H.S.) Colour Chart.
Origin: A tree of unknown origin, a Baby Dragon maple is similar to other Acer truncatum varieties except for its dwarf size, small leaves, thin willowy branches and overall weeping appearance. Its new growth also exhibits a distinctive, strong pink color (RHS 48C) throughout spring and summer. Baby Dragon maples are notably tolerant of both shaded and direct sun and heat conditions, and hardy to at least Zone 5 and possibly Zone 4. Baby Dragon maples also are prodigious leaf producers, which weighs down their thin, willowy branches. Its leaves are unusually small for Shantung maples, though proportionate to the dwarf size of the tree itself. Spring early flush leaf coloration exhibits deep pink (RHS 48A), rather than the normal reddish-purple (RHS reddish purple 60D) overtones of typical Acer truncatum. The coloration soon changes in early spring to deeper pink (RHS 48B). Summer growth has a distinctive, strong pink (RHS 48C) that contrasts noticeably with the mature leaves of green (RHS 140C), a contrast that persists until fall. Fall coloration is a consistent deeper yellow (RHS 4C) mixed with red (RHS 34C), whereas other Acer truncatum leaves are mostly yellow, with only occasional areas of lesser red or orange mixed with yellow, and seldom repeated in subsequent years. Mature leaves are green (RHS 140C), though not as dark green or glossy as typical Shantung. Leaf shape substantially matches the typical Acer truncatum profile but differs by having (a) bases cordate and only occasionally truncated, with five oblong-ovate lobes; (b) a deep cut two-thirds of way from tip to leaf base; and (c) a relatively large, central lobe that is broader at the bottom and triangular-ovate, producing an overall cordate-palmate leaf shape.
Classification: Acer truncatum ‘Baby Dragon’.
Parentage: Unknown; believed to be a seedling variation, mutation or adaptation of Acer truncatum.
Shape: Deciduous tree with rounded crown of uniform spread and many vigorous, crowded stems which are thin and willowy, resulting in a weeping appearance under the load of its profuse leafy foliage in spring and summer. See FIGS. 1A-1B. Caliper size is three (3 cm) centimeters inches in four (4) years, on grafts measured eighteen (18″) inches up from ground level.
Height: Four feet (4′-10″) ten inches (147.3 cm) tall for the mature, 17 year old tree of FIG. 1A. A three year graft was measured at four feet (4′-2″) two inches (127 cm) tall, indicating a very slow change in height despite vigorous annual new growth and leaf production, much smaller that typical of Acer truncatum. See FIGS. 1A-1B.
Spread: Six feet (6′-1″) one inch wide for the mature, 17 year old tree of FIG. 1A. A three year graft was measured at four feet (4′-9″) nine inches (144.8 cm) wide. This indicates a bushy, low growing dwarf variety that changes size very little despite vigorous new growth and leaf production, much less than typical of Acer truncatum. See FIGS. 1A-1B.
Trunk: Original trees have smooth, white bark due to their low caliper growth rate. See FIGS. 2A-2B. Grafted trees exhibit heavily fissured bark, with light brown raised areas and dark brown recessed areas, typical of the normal Shantung root stock. Caliper size of an original tree (the 17 year old tree of FIG. 1A) is four (4 cm) centimeters, while a three (3) year old graft already has a three (3 cm) centimeter caliper.
Branches: Noticeably thin and willowy, as well as closely and thickly branching. Current year wood starts as the same color as the leaves, but quickly becomes light gray. Late season current shoots can display a darker, red-brown and yellow-brown color in winter, similar to the leaf but darker. First and second year wood is pliable, becoming hard and dense in its third year. Lenticils are sparse, straw-colored and oval.
Leaves:
   Size.—Much smaller than other of Acer truncatum varieties; 20.2 (20.2 mm) millimeters long by 23.1 (23.1 mm) millimeters wide. Largest sampled: 33 mm by 40 mm. Smallest sampled: 8 mm by 10 mm.
   Shape.—Cordate base with only occasionally truncate base typical of Acer truncatum varieties. Glabrous with five (5) oblong-ovate lobes, deeply cut two-thirds of way from tip to leaf base. Margins entire and slightly wavy. Small, triangular teeth on central lobes, sometimes also found on one or more neighboring lobes. Central lobes are broader at the bottom and triangular-ovate. Acuminate apices and tufted, veined axils on the underside. Waxy cuticle typical of Acer truncatum creating a glossy shine at certain angles of sunlight, but not as dark green or shiny as Acer truncatum. Leaves are the same color on top and bottom, with prominent, raised veins on the bottom appearing disproportionate due to the small leaf size. See FIGS. 4A, 5B.
   Color.—Vibrant spring colors and summer foliage. Deep yellow and red fall colors. Colors on new growth are similar to normal Shantung maples except a more intense, deep pink and rare, strong pink in summer, in contrast to typical purplish-red Shantung colors. Early flush is deep pink (RHS 48A), turning in early spring to deeper pink (RHS 48B). See FIGS. 5A-5C. Summer growth is distinctive, strong pink (RHS 48C). See FIGS. 6A-6B. Fall colors become deep yellow (RHS 4C) to red (RHS 34C). See FIGS. 7A-7B. Hardened. mature growth foliage has a shiny green (RHS 140) on the leaf top with the same coloration on both the tops and bottoms of the leaves. Not as dark green or glossy as typical Shantung. See FIGS. 4A-4B.
Petiole: Slender, same red color as new growth, keeping a lighter red color on the sunny top side all summer. Small, averaging 17.7 mm in length, glabrous. Milky sap produced when broken.
Winter buds: Terminal buds maximum of three (3 mm) millimeters long by two (2 mm) millimeters wide, but can be much smaller; usually brown-gray. Current year shoots sometimes long but much thinner (3 mm) than normal Shantungs. Lateral buds very small (1 mm×0.5 mm). See FIG. 3A.
Flowering: None observed in 17 years. Expected to be nearly sterile although might flower and seed on grafted clones. If so, expected to be similar to or smaller than normal Shantung maples. Flowering season for Acer truncatum is spring (mid-March in Texas), before or simultaneously with leaves on terminal, typically forty by forty to sixty (40×40-60 mm) millimeter corymbose racemes having unequal, ten to twenty (10-20 mm.) millimeter length stems. Each 5-merous bloom is deep saucer-shaped, ten (10 mm) millimeters long and eight (8 mm) deep, with oblong to ovate petals and supported on one (1 cm) centimeter pedicels. Straw yellow (RHS 3C) color.
Reproductive organs: None observed in 17 years. Expected to be nearly sterile although might flower and seed on grafted clones. If so, expected to be similar to normal Shantung maples, though smaller, being imperfect; monoecious; trees first produce blooms of one gender, then of the other, then of the first again, often having both at the same time; approximately half of trees begin with each gender. Acer truncatum blooms have a central, circular, slightly lobed, straw yellow (RHS 3C) pistil with an ovary supported on two divergent stigma arms with short, one (1 mm) styles. Pistil is surrounded by eight to ten (8-10) one (1 mm) millimeter, glabrous stamens with numerous oval, two (2 mm) millimeter anthers on five to six (5-6 mm) millimeter filaments. In male flowers, filaments are longer, sometimes extending the anthers above the perianth, with a diminished pistil and vestigial ovary. Female blooms have shorter filaments wherein the smaller anthers are almost concealed within the perianth.
Pollen: None observed in 17 years. Expected to be nearly sterile although might flower and seed on grafted clones. If so, expected to be the same as other Acer truncatum varieties, having same color as flowers (straw yellow — RHS 3C).
Fruit: None observed in 17 years. Expected to be nearly sterile although might flower and seed on grafted clones. If so, expected to be similar to other Acer truncatum varieties though smaller, having pendulous, yellow-to-reddish, wide-angled samaras, with ovoid or obovoid, smooth and flattened nutlets ripening in the fall (October in Texas).
Disease resistance: No known problems.
Climate: Adapts well to any well-drained soil, even highly alkaline, clay soils. Very drought, wind and direct sunlight tolerant. Extremely hardy to at least Zone 5 and possibly to Zone 4 (USDA hardness guidelines). Baby Dragon maples have been observed on several occasions in full leaf in ambient temperatures of 24 degrees Fahrenheit without damage. More drought and sunshine tolerant than other Acer dwarf varieties, especially dwarf Japanese maple varieties, and it exhibits a good ability to keep healthy new growth without wilting under extreme heat and direct, intense sunlight conditions, as well as thriving in shady environments.
Comparison to other Acer truncatum varieties:
   Crown.—Similar rounded crown, but wider than height; expected height and spread much smaller.
   Trunk.—Smooth, white bark on original trees; grafted examples have heavily fissured trunk, with light brown raised areas between dark brown fissures, typical of normal Shantung root stock.
   Flowers.—Expected to be similar straw-yellow, identical in size.
   Reproductive organs.—Similar or smaller, monoecious imperfect, pollen same color as blooms.
   Fruit.—Expected to be similar or identical.
   Leaf shape and size.—Much smaller than Shantung leaves; average 2.0 cm long by 2.3 cm wide, compared with seven to thirteen (7-13 cm.) centimeters across for normal Shantung leaves. Only occasionally truncated, five oblong-ovate lobes; deep cut two-thirds of way from tip to leaf base; and a larger, central lobe that is broader at the bottom and triangular-ovate, producing an overall cordate-palmate leaf shape.
   Mature leaves.—Green (RHS 140C), though not as dark green or glossy as typical Shantung. Leaves are the same color on top and bottom, with prominent, raised veins on the bottom appearing disproportionate due to the small leaf size.
   Spring color.—Pink (RHS 48A), segueing into brighter pink (RHS 48B), in contrast to normal reddish-purple (RHS 60D) of normal Shantungs.
   Summer color.—Rare strong pink (RHS 48C) which persists throughout summer. Underlying mature, green leaves contrast with the pink, giving the tree a striking, pink haloed appearance throughout summer.
   Fall color.—Fall coloration is consistently a deep yellow (RHS 4C) mixed with red (RHS 34C), whereas other Acer truncatum leaves are mostly yellow, with only occasional areas of lesser red or orange mixed with yellow, and seldom repeated in subsequent years.
   Hardiness.—As drought and sunshine tolerant as larger Acer truncatum varieties, and more so than other especially dwarf maple varieties. Exhibits good ability to keep healthy new growth without wilting under extreme heat and direct, intense sunlight conditions. Seems to thrive equally well in sunshine or shade. Adapts well to any well-drained soil, even highly alkaline, clay soils. Very drought, wind and direct sunlight tolerant. Extremely hardy to at least Zone 5 and possibly to Zone 4 (USDA hardness guidelines).

I claim:

1. A new and distinct variety of an Acer truncatum maple tree named “Baby Dragon”, as described and illustrated herein.
